(19) United States
(12) Patent Application Publication
(10) Pub. No.: US 2025/0132037 A1
(43) Pub. Date: Apr. 24, 2025

(54) METHODS FOR PREDICTING CLINICAL IMPLICATIONS IN BREAST CANCER PATIENTS BASED ON TUMOR INFILTRATING LEUKOCYTES FRACTAL GEOMETRY

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: Edi Brogi, New York, NY (US); Larry Norton, New York, NY (US); Hannah Yong Wen, New York, NY (US); Jorge Sergio Reis, New York, NY (US); Matthew George Hanna, New York, NY (US)

(21) Appl. No.: 18/730,276

(22) PCT Filed: Jan. 19, 2023
(86) PCT No.: PCT/US2023/011149

§ 371 Date: Jul. 18, 2024

Related U.S. Application Data
(60) Provisional application No. 63/301,160, filed on Jan. 20, 2022.

(51) Int. Cl. G16H 50/20; G06T 7/00; G16H 20/10; G16H 20/40

(52) U.S. Cl. CPC G16H 50/20; G06T 7/0012; G16H 20/10; G16H 20/40; G06T 2207/20081; G06T 2207/30024; G06T 2207/30068; G06T 2207/30096

(57) ABSTRACT

Disclosed herein are systems and methods based on tumor infiltrating leukocyte (TIL) fractal geometry to predict clinical implications in breast cancer samples. Using machine learning techniques, an algorithmic classifier is constructed and trained on a cohort of tumor images (e.g. radiographic, immunohistochemical (IHC), and H&E images) to discriminate between TILs and cancer cells, and breast cancer samples are classified based on TIL fractal geometry/anatomic distribution in the tumor stroma. Fractal geometry-directed reassessment of classifications may prompt tumor type reclassification resulting in altered cancer therapy.

### CROSS-REFERENCE TO RELATED APPLICATIONS

This application claims the benefit of and priority to U.S. Provisional Application No. 63/301,160, filed Jan. 20, 2022, the disclosure of which is incorporated by reference herein in its entirety.

### TECHNICAL FIELD

Disclosed herein are systems and methods based on tumor infiltrating leukocyte (TIL) fractal geometry to predict clinical implications in breast cancer samples. Using machine learning techniques, an algorithmic classifier is constructed and trained on a cohort of tumor images (e.g. radiographic images, H&E images, immunohistochemical (IHC) images) to discriminate between TILs and cancer cells, and breast cancer samples are then classified based on TIL fractal geometry/anatomic distribution.

### BACKGROUND

The following description of the background of the present technology is provided simply as an aid in understanding the present technology and is not admitted to describe or constitute prior art to the present technology.

The fractal geometry implicit in nature illuminates many otherwise obscure relationships.¹ A natural phenomenon of interest in this regard is the infiltration of cancers with leukocytes, the effectors of the immune system. While in breast cancer the presence of tumor-infiltrating leukocytes (TILs) conveys prognostic and predictive information, the mechanisms remain conjectural.² Nevertheless, this phenomenon may have important implications with respect to immune-directed therapy.³ Accordingly, theoretic considerations have focused largely on potentially manipulatable viability-related interactions-both positive and negative-between the cancer cells and the TILs.⁴ Useful, but complex, computationally intensive, and often non-intuitive (i.e. computer machine learning) approaches to this subject have been proposed.⁵

### SUMMARY OF THE PRESENT TECHNOLOGY

In one aspect, the present disclosure provides a method for predicting prognosis in a breast cancer patient comprising applying, by a computing system having one or more processors, a trained model to detect and quantify (a) cancer cells and (b) tumor-infiltrating leukocytes (TILs) in a biomedical image of a resected tumor sample including a tumor stromal region, wherein the resected tumor sample is obtained from the breast cancer patient; computing a fractal-geometric metric based on anatomic distribution of the TILs in the biomedical image of the resected tumor sample; and determining that the breast cancer patient has a favorable prognosis when the fractal-geometric metric falls below a predetermined threshold, or determining that the breast cancer patient has a negative prognosis when the fractal-geometric metric is at or above a predetermined threshold. In some embodiments, negative prognosis comprises recurrent disease in breast tissue, bone tissue, or brain tissue. Additionally or alternatively, in some embodiments, the method further comprises providing a cancer therapy recommendation based on the fractal-geometric metric, and/or administering a cancer therapy to the breast cancer patient based on the fractal-geometric metric. Examples of suitable cancer therapy may comprise one or more of surgery, chemotherapy, immunotherapy and radiation therapy.

In one aspect, the present disclosure provides a method for selecting a breast cancer patient for treatment with a chemotherapeutic agent comprising applying, by a computing system having one or more processors, a trained model to detect and quantify (a) cancer cells and (b) tumor-infiltrating leukocytes (TILs) in a biomedical image of a resected tumor sample including a tumor stromal region, wherein the resected tumor sample is obtained from the breast cancer patient; computing a fractal-geometric metric based on anatomic distribution of the TILs in the biomedical image of the resected tumor sample; and administering a chemotherapeutic agent to the breast cancer patient, wherein the fractal-geometric metric of the breast cancer patient falls below a predetermined threshold. Examples of chemotherapeutic agents include, but are not limited to, alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, and endocrine/hormonal agents. In certain embodiments, the chemotherapeutic agent is selected from the group consisting of cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), bevacizumab, oxaliplatin, melphalan, etoposide, mechlorethamine, bleomycin, microtubule poisons, annonaceous acetogenins, or combinations thereof.

In another aspect, the present disclosure provides a method for selecting a breast cancer patient for surgery, radiation therapy, or immunotherapy comprising applying, by a computing system having one or more processors, a trained model to detect and quantify (a) cancer cells and (b) tumor-infiltrating leukocytes (TILs) in a biomedical image of a resected tumor sample including a tumor stromal region, wherein the resected tumor sample is obtained from the breast cancer patient; computing a fractal-geometric metric based on anatomic distribution of the TILs in the biomedical image of the resected tumor sample; and administering surgery, radiation therapy, or immunotherapy to the breast cancer patient, wherein the fractal-geometric metric of the breast cancer patient is at or above a predetermined threshold. In certain embodiments, the immunotherapy comprises one or more of an anti-PD-1 antibody, an anti-PD-L1 antibody, an anti-PD-L2 antibody, an anti-CTLA-4 antibody, an anti-TIM3 antibody, an anti-TIGIT antibody, an anti-VISTA antibody, an anti-B7-H3 antibody, an anti-BTLA antibody, an anti-CD73 antibody, or an anti-LAG-3 antibody. Examples of immunotherapy may include one or more of pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189.

Additionally or alternatively, in some embodiments, the breast cancer is triple negative breast cancer, HER2-positive breast cancer, Estrogen-Receptor (ER) positive breast cancer, or Progesterone-Receptor (PR) positive breast cancer. The breast cancer may be metastatic or primary. In certain embodiments, the breast cancer patient suffers from stage I cancer, stage II cancer, stage III cancer, or stage IV breast cancer. Additionally or alternatively, in certain embodiments, the biomedical image is a hematoxylin and eosin (H&E)-stained image, a radiographic image or an immunohistochemical (IHC) image. In any of the preceding embodiments of the methods disclosed herein, the tumor stromal region includes basement membrane, fibroblasts, extracellular matrix, immune cells, and mesenchymal stromal cells.

Additionally or alternatively, in some embodiments, the methods of the present technology further comprise computing an additional metric based on quantity and anatomic distribution of HER2-expressing cancer cells, and/or Trop2-expressing cancer cells in the biomedical image of the resected tumor sample.

In any and all embodiments of the methods disclosed herein, the trained model is a machine learning model generated using a machine learning technique. In some embodiments, the machine learning technique is a random forest technique, and the machine learning classification model is a random forest model. Alternatively, other machine learning classification models such as Naïve Bays, Support Vector Machines, Decision Trees, KNN (k-nearest neighbours), Generalized Linear Models, Bagging, convolutional neural networks (CNN) and the like may be used.

In one aspect, the present disclosure provides a computer system for predicting prognosis in a breast cancer patient, the computing system comprising a processor and a memory with instructions which, when executed by the processor, cause the processor to: apply, by a computing system having one or more processors, a trained model to detect and quantify (a) cancer cells and (b) tumor-infiltrating leukocytes (TILs) in a biomedical image of a resected tumor sample including a tumor stromal region, wherein the resected tumor sample is obtained from the patient; compute a fractal-geometric metric based on anatomic distribution of the TILs in the biomedical image of the resected tumor sample; and determine that the breast cancer patient has a favorable prognosis when the fractal-geometric metric falls below a predetermined threshold, or determining that the breast cancer patient has a negative prognosis when the fractal-geometric metric is at or above a predetermined threshold. In some embodiments, negative prognosis comprises recurrent disease in breast tissue, bone tissue, or brain tissue.

Additionally or alternatively, in some embodiments, the breast cancer is triple negative breast cancer, HER2-positive breast cancer, Estrogen-Receptor (ER) positive breast cancer, or Progesterone-Receptor (PR) positive breast cancer. The breast cancer may be metastatic or primary. In certain embodiments, the breast cancer patient suffers from stage I cancer, stage II cancer, stage III cancer, or stage IV breast cancer. Additionally or alternatively, in certain embodiments, the biomedical image is a hematoxylin and eosin (H&E)-stained image, a radiographic image or an immunohistochemical (IHC) image. In any of the preceding embodiments of the methods disclosed herein, the tumor stromal region includes basement membrane, fibroblasts, extracellular matrix, immune cells, and mesenchymal stromal cells.

In any and all embodiments of the computer systems disclosed herein, the trained model is a machine learning model generated using a machine learning technique. In some embodiments, the machine learning technique is a random forest technique, and the machine learning classification model is a random forest model. Alternatively, other machine learning classification models such as Naïve Bays, Support Vector Machines, Decision Trees, KNN (k-nearest neighbours), Generalized Linear Models, Bagging, convolutional neural networks (CNN) and the like may be used.

Additionally or alternatively, in some embodiments of the computer systems disclosed herein, the instructions further cause the processor to compute an additional metric based on quantity and anatomic distribution of HER2-expressing cancer cells, and/or Trop2-expressing cancer cells in the biomedical image of the resected tumor sample. In certain embodiments, of the computer systems disclosed herein, the instructions further cause the processor to provide a cancer therapy recommendation based on the fractal-geometric metric. Examples of cancer therapy include surgery, chemotherapy, immunotherapy and radiation therapy.

### BRIEF DESCRIPTION OF THE DRAWINGS

FIG. 1A is a block diagram depicting an embodiment of a network environment comprising a client device in communication with server device.

FIG. 1B is a block diagram depicting a cloud computing environment comprising client device in communication with cloud service providers.

FIGS. 1C and 1D are block diagrams depicting embodiments of computing devices useful in connection with the methods and systems described herein.

FIG. 2 depicts a system that includes a computing device and an image processing system for biological samples according to various potential embodiments.

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

The present disclosure is directed to the application of leukocyte-cancer fractal geometry to determine fundamental aspects of tumor initiation and growth. To complement existing computer machine learning approaches, the present disclosure provides a metric with several characteristics: simplicity, ease of assessment, clinical relevance, and interpretability. Fractal geometry appears to be germane in that cancers grow by an iterative process and are hence anatomically irregular but with the same degree of irregularity on all scales. If leukocyte distribution reflects this growth pattern, then the number of leukocytes in a microscopic area should be proportional to the length of that area raised to a power expressing the dimensionality of the process.⁶

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

The terms “cancer” or “tumor” are used interchangeably and refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. As used herein, the term “cancer” includes premalignant, as well as malignant cancers. In some embodiments, the cancer is colorectal cancer, lung cancer, breast cancer, ovarian cancer, uterine cancer, or thyroid cancer.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “overall survival” or “OS” means the observed length of life from the start of treatment to death or the date of last contact.

As used herein, “progression free survival” or “PFS” is the time from treatment to the date of the first confirmed disease progression per RECIST 1.1 and immune-related RECIST (irRECIST) criteria.

“RECIST” shall mean an acronym that stands for “Response Evaluation Criteria in Solid Tumors” and is a set of published rules that define when cancer patients improve (“respond”), stay the same (“stable”) or worsen (“progression”) during treatments. Response as defined by RECIST criteria have been published, for example, at Journal of the National Cancer Institute, Vol. 92, No. 3, Feb. 2, 2000 and RECIST criteria can include other similar published definitions and rule sets. One skilled in the art would understand definitions that go with RECIST criteria, as used herein, such as “Partial Response (PR),” “Complete Response (CR),” “Stable Disease (SD)” and “Progressive Disease (PD).”

The irRECIST overall tumor assessment is based on total measurable tumor burden (TMTB) of measured target and new lesions, non-target lesion assessment and new non-measurable lesions. At baseline, the sum of the longest diameters (SumD) of all target lesions (up to 2 lesions per organ, up to total 5 lesions) is measured. At each subsequent tumor assessment (TA), the SumD of the target lesions and of new, measurable lesions (up to 2 new lesions per organ, total 5 new lesions) are added together to provide the TMTB.

Overall Assessments by irRECIST

| | |
|---|---|
| Complete Response (irCR) | Complete disappearance of all measurable and non- |
| | measurable lesions. Lymph nodes must decrease to <10 |
| | mm in short axis. |
| Partial Response (irPR) | Decrease of ≥30% in TMTB relative to baseline, non-target |
| | lesions are irNN, and no unequivocal progression of new |
| | nonmeasurable lesions |
| | If new measurable lesions appear in subjects with no |
| | target lesions at baseline, irPD will be assessed. That irPD |
| | time point will be considered a new baseline, and all |
| | subsequent time points will be compared to it for response |
| | assessment. irPR is possible if the TMTB of new |
| | measurable lesions decreases by ≥30% compared to the |
| | first irPD documentation |
| | irRECIST can be used in the adjuvant setting, in |
| | subjects with no visible disease on CT/MRI scans. The |
| | appearance of new measurable lesion(s) automatically leads |
| | to an increase in TMTB by 100% and leads to irPD. These |
| | subjects can achieve a response if the TMTB decreases at |
| | follow-up, as a sign of delayed response. |
| | Based on the above, sponsors may consider |
| | enrolling subjects with no measurable disease and/or no |
| | visible disease in studies with response related endpoints. |
| Stable Disease (irSD) | Failure to meet criteria for irCR or irPR in the absence of |
| | irPD |
| Progressive Disease (irPD) | Minimum 20% increase and minimum 5 mm absolute |
| | increase in TMTB compared to nadir, or irPD for non-target |
| | or new non-measurable lesions. Confirmation of |
| | progression is recommended minimum 4 weeks after the |
| | first irPD assessment. An irPD confirmation scan may be |
| | recommended for subjects with a minimal TMTB %- |
| | increase over 20% and especially during the flare time- |
| | window of the first 12 weeks of treatment, depending on the |
| | compound efficacy expectations, to account for expected |
| | delayed response. |
| | In irRECIST a substantial and unequivocal increase |
| | of non-target lesions is indicative of progression. |
| | IrPD may be assigned for a subject with multiple |
| | new non-measurable lesions if they are considered to be a |
| | sign of unequivocal massive worsening |
| Other | irNE: used in exceptional cases where insufficient data |
| | exist. |
| | irND: in adjuvant setting when no disease is detected |
| | irNN:, no target disease was identified at baseline, and at |
| | follow-up the subject fails to meet criteria for irCR or irPD |

As used herein, the terms “subject”, “patient”, or “individual” can be an individual organism, a vertebrate, a mammal, or a human. In some embodiments, the subject, patient or individual is a human.

As used herein, “survival” refers to the subject remaining alive, and includes overall survival as well as progression free survival.

### Systems, Devices, and Methods for Classifying Breast Cancer Tumor Samples

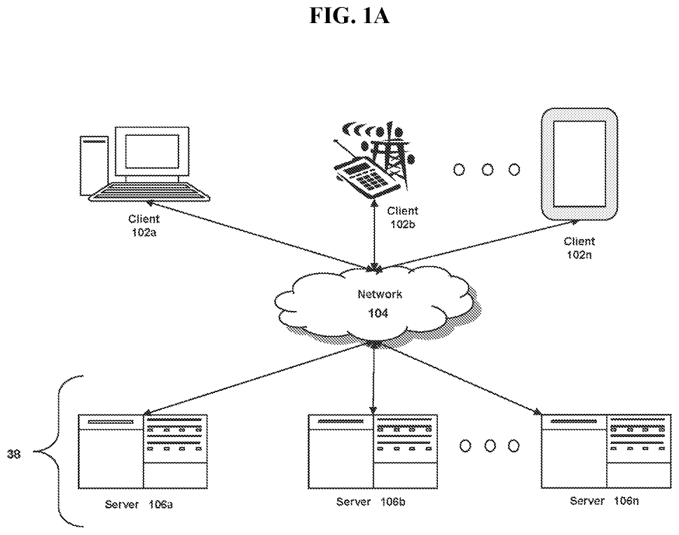

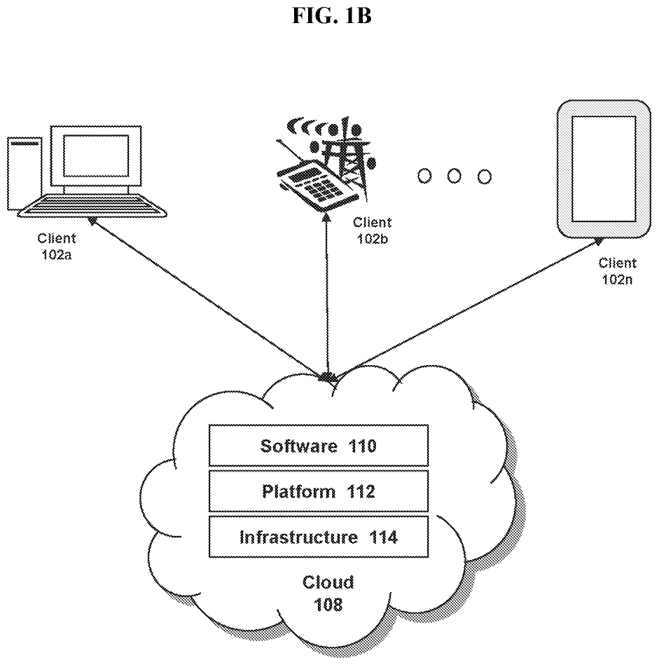

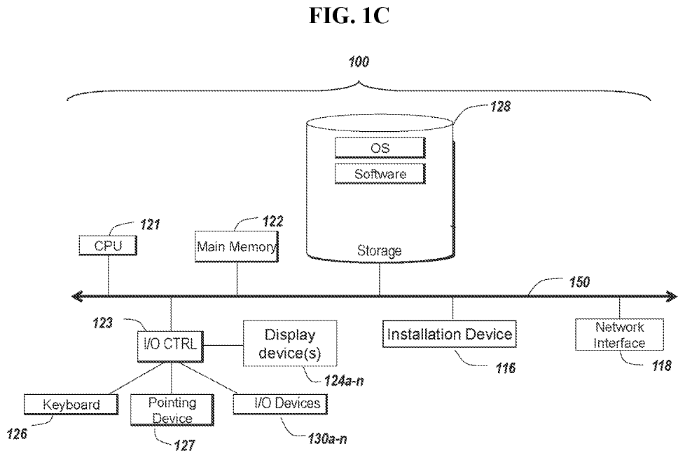

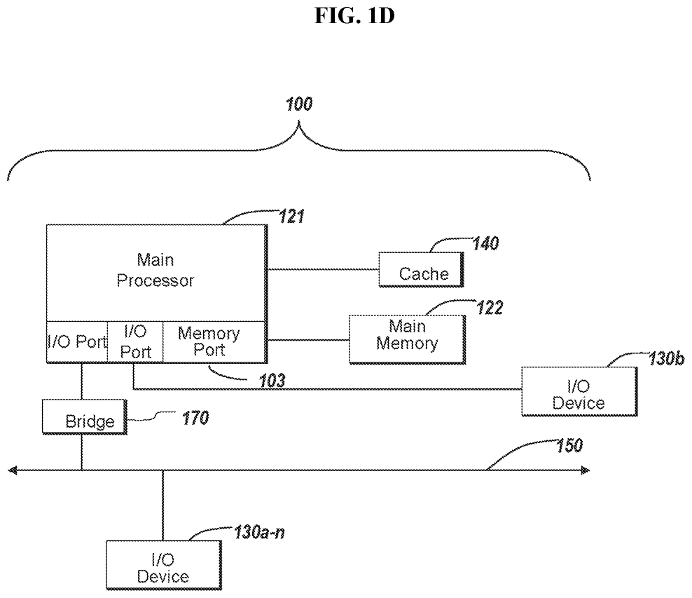

Aspects of the operating environment as well as associated system components (e.g., hardware elements) in connection with various embodiments of the methods and systems described herein will now be discussed. Referring to FIG. 1A, an embodiment of a network environment is depicted. In brief overview, the illustrated exploring network environment includes one or more clients 102 a-102 n (also generally referred to as local machine(s) 102, client(s) 102, client node(s) 102, client machine(s) 102, client computer(s) 102, client device(s) 102, endpoint(s) 102, or endpoint node(s) 102) in communication with one or more servers 106 a-106 n (also generally referred to as server(s) 106, node 106, or remote machine(s) 106) via one or more networks 104. In some embodiments, a client 102 has the capacity to function as both a client node seeking access to resources provided by a server and as a server providing access to hosted resources for other clients 102 a-102 n.

Although FIG. 1A shows a network 104 between the clients 102 and the servers 106, the clients 102 and the servers 106 may be on the same network 104. In some embodiments, there are multiple networks 104 between the clients 102 and the servers 106. In one of these embodiments, a network 104′ (not shown) may be a private network and a network 104 may be a public network. In another of these embodiments, a network 104 may be a private network and a network 104′ a public network. In still another of these embodiments, networks 104 and 104′ may both be private networks.

The network 104 may be connected via wired or wireless links. Wired links may include Digital Subscriber Line (DSL), coaxial cable lines, or optical fiber lines. The wireless links may include BLUETOOTH, Wi-Fi, NFC, RFID Worldwide Interoperability for Microwave Access (WiMAX), an infrared channel or satellite band. The wireless links may also include any cellular network standards used to communicate among mobile devices, including standards that qualify as 1G, 2G, 3G, or 4G. The network standards may qualify as one or more generation of mobile telecommunication standards by fulfilling a specification or standards such as the specifications maintained by International Telecommunication Union. The 3G standards, for example, may correspond to the International Mobile Telecommunications-2000 (IMT-2000) specification, and the 4G standards may correspond to the International Mobile Telecommunications Advanced (IMT-Advanced) specification. Examples of cellular network standards include AMPS, GSM, GPRS, UMTS, LTE, LTE Advanced, Mobile WiMAX, and WiMAX-Advanced. Cellular network standards may use various channel access methods e.g. FDMA, TDMA, CDMA, or SDMA. In some embodiments, different types of data may be transmitted via different links and standards. In other embodiments, the same types of data may be transmitted via different links and standards.

The network 104 may be any type and/or form of network. The geographical scope of the network 104 may vary widely and the network 104 can be a body area network (BAN), a personal area network (PAN), a local-area network (LAN), e.g. Intranet, a metropolitan area network (MAN), a wide area network (WAN), or the Internet. The topology of the network 104 may be of any form and may include, e.g., any of the following: point-to-point, bus, star, ring, mesh, or tree. The network 104 may be an overlay network, which is virtual and sits on top of one or more layers of other networks 104′. The network 104 may be of any such network topology as known to those ordinarily skilled in the art capable of supporting the operations described herein. The network 104 may utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPv6), or the link layer. The network 104 may be a type of a broadcast network, a telecommunications network, a data communication network, or a computer network.

In some embodiments, the system may include multiple, logically-grouped servers 106. In one of these embodiments, the logical group of servers may be referred to as a server farm or a machine farm. In another of these embodiments, the servers 106 may be geographically dispersed. In other embodiments, a machine farm 38 may be administered as a single entity. In still other embodiments, the machine farm 38 includes a plurality of machine farms 38. The servers 106 within each machine farm 38 can be heterogeneous—one or more of the servers 106 or machines 106 can operate according to one type of operating system platform (e.g., WINDOWS NT, manufactured by Microsoft Corp. of Redmond, Wash.), while one or more of the other servers 106 can operate on according to another type of operating system platform (e.g., Unix, Linux, or Mac OS X).

In one embodiment, servers 106 in the machine farm 38 may be stored in high-density rack systems, along with associated storage systems, and located in an enterprise data center. In this embodiment, consolidating the servers 106 in this way may improve system manageability, data security, the physical security of the system, and system performance by locating servers 106 and high performance storage systems on localized high performance networks. Centralizing the servers 106 and storage systems and coupling them with advanced system management tools allows more efficient use of server resources.

The servers 106 of each machine farm 38 do not need to be physically proximate to another server 106 in the same machine farm. Thus, the group of servers 106 logically grouped as a machine farm may be interconnected using a wide-area network (WAN) connection or a metropolitan-area network (MAN) connection. For example, a machine farm 38 may include servers 106 physically located in different continents or different regions of a continent, country, state, city, campus, or room. Data transmission speeds between servers 106 in the machine farm 38 can be increased if the servers 106 are connected using a local-area network (LAN) connection or some form of direct connection. Additionally, a heterogeneous machine farm 38 may include one or more servers 106 operating according to a type of operating system, while one or more other servers 106 execute one or more types of hypervisors rather than operating systems. In these embodiments, hypervisors may be used to emulate virtual hardware, partition physical hardware, virtualized physical hardware, and execute virtual machines that provide access to computing environments, allowing multiple operating systems to run concurrently on a host computer. Native hypervisors may run directly on the host computer. Hypervisors may include VMware ESX/ESXi, manufactured by VMWare, Inc., of Palo Alto, Calif.; the Xen hypervisor, an open source product whose development is overseen by Citrix Systems, Inc.; the HYPER-V hypervisors provided by Microsoft or others. Hosted hypervisors may run within an operating system on a second software level. Examples of hosted hypervisors may include VMware Workstation and VIRTUALBOX.

Management of the machine farm 38 may be de-centralized. For example, one or more servers 106 may comprise components, subsystems and modules to support one or more management services for the machine farm 38. In one of these embodiments, one or more servers 106 provide functionality for management of dynamic data, including techniques for handling failover, data replication, and increasing the robustness of the machine farm 38. Each server 106 may communicate with a persistent store and, in some embodiments, with a dynamic store.

Server 106 may be a file server, application server, web server, proxy server, appliance, network appliance, gateway, gateway server, virtualization server, deployment server, SSL VPN server, or firewall. In one embodiment, the server 106 may be referred to as a remote machine or a node. In another embodiment, a plurality of nodes may be in the path between any two communicating servers.

Referring to FIG. 1B, a cloud computing environment is depicted. A cloud computing environment may provide client 102 with one or more resources provided by a network environment. The cloud computing environment may include one or more clients 102 a-102n, in communication with the cloud 108 over one or more networks 104. Clients 102 may include, e.g., thick clients, thin clients, and zero clients. A thick client may provide at least some functionality even when disconnected from the cloud 108 or servers 106. A thin client or a zero client may depend on the connection to the cloud 108 or server 106 to provide functionality. A zero client may depend on the cloud 108 or other networks 104 or servers 106 to retrieve operating system data for the client device. The cloud 108 may include back end platforms, e.g., servers 106, storage, server farms or data centers.

The cloud 108 may be public, private, or hybrid. Public clouds may include public servers 106 that are maintained by third parties to the clients 102 or the owners of the clients. The servers 106 may be located off-site in remote geographical locations as disclosed above or otherwise. Public clouds may be connected to the servers 106 over a public network. Private clouds may include private servers 106 that are physically maintained by clients 102 or owners of clients. Private clouds may be connected to the servers 106 over a private network 104. Hybrid clouds 108 may include both the private and public networks 104 and servers 106.

The cloud 108 may also include a cloud based delivery, e.g. Software as a Service (SaaS) 110, Platform as a Service (PaaS) 112, and Infrastructure as a Service (IaaS) 114. IaaS may refer to a user renting the use of infrastructure resources that are needed during a specified time period. IaaS providers may offer storage, networking, servers or virtualization resources from large pools, allowing the users to quickly scale up by accessing more resources as needed. PaaS providers may offer functionality provided by IaaS, including, e.g., storage, networking, servers or virtualization, as well as additional resources such as, e.g., the operating system, middleware, or runtime resources. Examples of PaaS include WINDOWS AZURE provided by Microsoft Corporation of Redmond, Wash., Google App Engine provided by Google Inc., and HEROKU provided by Heroku, Inc. of San Francisco, Calif. SaaS providers may offer the resources that PaaS provides, including storage, networking, servers, virtualization, operating system, middleware, or runtime resources. In some embodiments, SaaS providers may offer additional resources including, e.g., data and application resources.

Clients 102 may access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards may allow clients access to resources over HTTP, and may use Representational State Transfer (REST) protocol or Simple Object Access Protocol (SOAP). Clients 102 may access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that may be built on REST, HTTP, XML, or other protocols. Clients 102 may access SaaS resources through the use of web-based user interfaces, provided by a web browser. Clients 102 may also access SaaS resources through smartphone or tablet applications, including. Clients 102 may also access SaaS resources through the client operating system.

In some embodiments, access to IaaS, PaaS, or SaaS resources may be authenticated. For example, a server or authentication server may authenticate a user via security certificates, HTTPS, or API keys. API keys may include various encryption standards such as, e.g., Advanced Encryption Standard (AES). Data resources may be sent over Transport Layer Security (TLS) or Secure Sockets Layer (SSL).

The client 102 and server 106 may be deployed as and/or executed on any type and form of computing device, e.g. a computer, network device or appliance capable of communicating on any type and form of network and performing the operations described herein. FIGS. 1C and 1D depict block diagrams of a computing device 100 useful for practicing an embodiment of the client 102 or a server 106. As shown in FIGS. 1C and 1D, each computing device 100 includes a central processing unit 121, and a main memory unit 122. As shown in FIG. 1C, a computing device 100 may include a storage device 128, an installation device 116, a network interface 118, an I/O controller 123, display devices 124a-124n, a keyboard 126 and a pointing device 127, e.g. a mouse. The storage device 128 may include, without limitation, an operating system, and/or software 120. As shown in FIG. 1D, each computing device 100 may also include additional optional elements, e.g. a memory port 103, a bridge 170, one or more input/output devices 130a-130n (generally referred to using reference numeral 130), and a cache memory 140 in communication with the central processing unit 121.

The central processing unit 121 is any logic circuitry that responds to and processes instructions fetched from the main memory unit 122. In many embodiments, the central processing unit 121 is provided by a microprocessor unit. The computing device 100 may be based on any of these processors, or any other processor capable of operating as described herein. The central processing unit 121 may utilize instruction level parallelism, thread level parallelism, different levels of cache, and multi-core processors. A multi-core processor may include two or more processing units on a single computing component.

Main memory unit 122 may include one or more memory chips capable of storing data and allowing any storage location to be directly accessed by the microprocessor 121. Main memory unit 122 may be volatile and faster than storage 128 memory. Main memory units 122 may be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 122 or the storage 128 may be non-volatile; e.g., non-volatile read access memory (NVRAIVI), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 122 may be based on any of the above described memory chips, or any other available memory chips capable of operating as described herein. In the embodiment shown in FIG. 1C, the processor 121 communicates with main memory 122 via a system bus 150 (described in more detail below). FIG. 1D depicts an embodiment of a computing device 100 in which the processor communicates directly with main memory 122 via a memory port 103. For example, in FIG. 1D the main memory 122 may be DRDRAM.

FIG. 1D depicts an embodiment in which the main processor 121 communicates directly with cache memory 140 via a secondary bus, sometimes referred to as a backside bus. In other embodiments, the main processor 121 communicates with cache memory 140 using the system bus 150. Cache memory 140 typically has a faster response time than main memory 122 and is typically provided by SRAM, BSRAM, or EDRAM. In the embodiment shown in FIG. 1D, the processor 121 communicates with various I/O devices 130 via a local system bus 150. Various buses may be used to connect the central processing unit 121 to any of the I/O devices 130, including a PCI bus, a PCI-X bus, or a PCI-Express bus, or a NuBus. For embodiments in which the I/O device is a video display 124, the processor 121 may use an Advanced Graphics Port (AGP) to communicate with the display 124 or the I/O controller 123 for the display 124. FIG. 1D depicts an embodiment of a computer 100 in which the main processor 121 communicates directly with I/O device 130b or other processors 121′ via HYPERTRANSPORT, RAPIDIO, or INFINIBAND communications technology. FIG. 1D also depicts an embodiment in which local busses and direct communication are mixed: the processor 121 communicates with I/O device 130a using a local interconnect bus while communicating with I/O device 130b directly.

A wide variety of I/O devices 130a-130n may be present in the computing device 100. Input devices may include keyboards, mice, trackpads, trackballs, touchpads, touch mice, multi-touch touchpads and touch mice, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, and 3D printers.

Devices 130a-130n may include a combination of multiple input or output devices, including. Some devices 130a-130n allow gesture recognition inputs through combining some of the inputs and outputs. Some devices 130a-130n provides for facial recognition which may be utilized as an input for different purposes including authentication and other commands. Some devices 130a-130n provides for voice recognition and inputs. Additional devices 130a-130n have both input and output capabilities, including, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures. Some touchscreen devices, including, such as on a table-top or on a wall, and may also interact with other electronic devices. Some I/O devices 130a-130n, display devices 124a-124n or group of devices may be augment reality devices. The I/O devices may be controlled by an I/O controller 123 as shown in FIG. 1C. The I/O controller may control one or more I/O devices, such as, e.g., a keyboard 126 and a pointing device 127, e.g., a mouse or optical pen. Furthermore, an I/O device may also provide storage and/or an installation medium 116 for the computing device 100. In still other embodiments, the computing device 100 may provide USB connections (not shown) to receive handheld USB storage devices. In further embodiments, an I/O device 130 may be a bridge between the system bus 150 and an external communication bus, e.g. a USB bus, a SCSI bus, a FireWire bus, an Ethernet bus, a Gigabit Ethernet bus, a Fibre Channel bus, or a Thunderbolt bus.

In some embodiments, display devices 124a-124n may be connected to I/O controller 123. Display devices may include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays may use, e.g. stereoscopy, polarization filters, active shutters, or autostereoscopy. Display devices 124a-124n may also be a head-mounted display (HMD). In some embodiments, display devices 124a-124n or the corresponding I/O controllers 123 may be controlled through or have hardware support for OPENGL or DIRECTX API or other graphics libraries.

In some embodiments, the computing device 100 may include or connect to multiple display devices 124a-124n, which each may be of the same or different type and/or form. As such, any of the I/O devices 130a-130n and/or the I/O controller 123 may include any type and/or form of suitable hardware, software, or combination of hardware and software to support, enable or provide for the connection and use of multiple display devices 124a-124n by the computing device 100. For example, the computing device 100 may include any type and/or form of video adapter, video card, driver, and/or library to interface, communicate, connect or otherwise use the display devices 124a-124n. In one embodiment, a video adapter may include multiple connectors to interface to multiple display devices 124a-124n. In other embodiments, the computing device 100 may include multiple video adapters, with each video adapter connected to one or more of the display devices 124a-124n. In some embodiments, any portion of the operating system of the computing device 100 may be configured for using multiple displays 124a-124n. In other embodiments, one or more of the display devices 124a-124n may be provided by one or more other computing devices 100a or 100 b connected to the computing device 100, via the network 104. In some embodiments software may be designed and constructed to use another computer's display device as a second display device 124a for the computing device 100.

Referring again to FIG. 1C, the computing device 100 may comprise a storage device 128 (e.g. one or more hard disk drives or redundant arrays of independent disks) for storing an operating system or other related software, and for storing application software programs such as any program related to the software 120. Examples of storage device 128 include, e.g., hard disk drive (HDD); optical drive; solid-state drive (SSD); USB flash drive; or any other device suitable for storing data. Some storage devices may include multiple volatile and non-volatile memories, including, e.g., solid state hybrid drives that combine hard disks with solid state cache. Some storage device 128 may be non-volatile, mutable, or read-only. Some storage device 128 may be internal and connect to the computing device 100 via a bus 150. Some storage device 128 may be external and connect to the computing device 100 via an I/O device 130 that provides an external bus. Some storage device 128 may connect to the computing device 100 via the network interface 118 over a network 104. Some client devices 100 may not require a non-volatile storage device 128 and may be thin clients or zero clients 102. Some storage device 128 may also be used as an installation device 116, and may be suitable for installing software and programs.

Client device 100 may also install software or application from an application distribution platform. An application distribution platform may facilitate installation of software on a client device 102. An application distribution platform may include a repository of applications on a server 106 or a cloud 108, which the clients 102a-102n may access over a network 104. An application distribution platform may include application developed and provided by various developers. A user of a client device 102 may select, purchase and/or download an application via the application distribution platform.

Furthermore, the computing device 100 may include a network interface 118 to interface to the network 104 through a variety of connections including, but not limited to, standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). In one embodiment, the computing device 100 communicates with other computing devices 100′ via any type and/or form of gateway or tunneling protocol e.g. Secure Socket Layer (SSL) or Transport Layer Security (TLS). The network interface 118 may comprise a built-in network adapter, network interface card, PCMCIA network card, EXPRESSCARD network card, card bus network adapter, wireless network adapter, USB network adapter, modem or any other device suitable for interfacing the computing device 100 to any type of network capable of communication and performing the operations described herein.

A computing device 100 of the sort depicted in FIGS. 1B and 1C may operate under the control of an operating system, which controls scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 2000, WINDOWS Server 2012, WINDOWS CE, WINDOWS Phone, WINDOWS XP, WINDOWS VISTA, and WINDOWS 7, WINDOWS RT, and WINDOWS 8 all of which are manufactured by Microsoft Corporation of Redmond, Wash.; MAC OS and iOS, manufactured by Apple, Inc. of Cupertino, Calif.; and Linux, a freely-available operating system, e.g. Linux Mint distribution (“distro”) or Ubuntu, distributed by Canonical Ltd. of London, United Kingdom; or Unix or other Unix-like derivative operating systems; and Android, designed by Google, of Mountain View, Calif., among others. Some operating systems, including, e.g., the CHROME OS by Google, may be used on zero clients or thin clients, including, e.g., CHROMEBOOKS.

The computer system 100 can be any workstation, telephone, desktop computer, laptop or notebook computer, netbook, tablet, server, handheld computer, mobile telephone, smartphone or other portable telecommunications device, media playing device, a gaming system, mobile computing device, or any other type and/or form of computing, telecommunications or media device that is capable of communication. The computer system 100 has sufficient processor power and memory capacity to perform the operations described herein. In some embodiments, the computing device 100 may have different processors, operating systems, and input devices consistent with the device.

In some embodiments, the computing device 100 is a gaming system. In some embodiments, the computing device 100 is a digital audio player. Some digital audio players may have other functionality, including, e.g., a gaming system or any functionality made available by an application from a digital application distribution platform. In some embodiments, the computing device 100 is a portable media player or digital audio player supporting file formats including. In some embodiments, the computing device 100 is a tablet. In other embodiments, the computing device 100 is an eBook reader. In some embodiments, the communications device 102 includes a combination of devices, e.g. a smartphone combined with a digital audio player or portable media player. For example, one of these embodiments is a smartphone. In yet another embodiment, the communications device 102 is a laptop or desktop computer equipped with a web browser and a microphone and speaker system, e.g. a telephony headset. In these embodiments, the communications devices 102 are web-enabled and can receive and initiate phone calls. In some embodiments, a laptop or desktop computer is also equipped with a webcam or other video capture device that enables video chat and video call. In some embodiments, the communication device 102 is a wearable mobile computing device.

In some embodiments, the status of one or more machines 102, 106 in the network 104 is monitored, generally as part of network management. In one of these embodiments, the status of a machine may include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

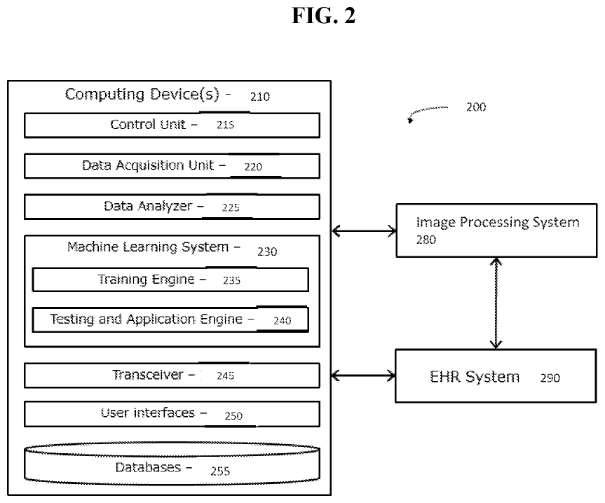

Referring to FIG. 2, in various embodiments, a system 200 may include a computing device 210 (or multiple computing devices, co-located or remote to each other), an image processing system for biological samples 280, and an electronic health record (EHR) system 290. In various embodiments, computing device 210 (or components thereof) may be integrated with the image processing system for biological samples 280 (or components thereof) and/or EHR system 290 (or components thereof). In various embodiments, the image processing system for biological samples 280 may include, may be, or may employ, radiographic images, immunohistochemical (IHC) images, and H&E images on solid tumor samples.

In various embodiments, the EHR system 290 may include, may be, or may employ, various computing devices that include health records of patients and study subjects (including devices of hospitals, clinics, healthcare practitioners, etc.), obtained from various sources, such as entries by healthcare practitioners, image processing system for biological samples 280, university and hospital systems, government agency systems, etc.

In various embodiments, the computing device 210 (or multiple computing devices) may be used to control, and receive signals acquired via, components of image processing system for biological samples 280. The computing device 210 may include one or more processors and one or more volatile and non-volatile memories for storing computing code and data that are captured, acquired, recorded, and/or generated. The computing device 210 may include a control unit 215 that in certain embodiments may be configured to exchange control signals with image processing system for biological samples 280, allowing the computing device 210 to be used to control, for example, processing of samples and/or delivery of data generated and/or acquired through processing of samples.

In various embodiments, computing device 210 may include a data acquisition unit 220 that may be configured to exchange control signals, or otherwise communicate, with image processing system for biological samples 280 (or components thereof) and/or EHR system 290, allowing the computing device 210 to be used to control the capture of physiological data and/or signals via sensors of the image processing system for biological samples 280, retrieve data or signals (e.g., from image processing system for biological samples 280, EHR system 290, and/or memory devices where data is stored), and direct transfer of data or signals (e.g., to image processing system for biological samples 280 as feedback thereto, to EHR system 290, to memory for storage, and/or to other systems or devices).

In various embodiment, a data analyzer 225 may direct analysis of the data and signals, and output analysis results. Data analyzer 225 may be used, for example, to transform raw data captured or obtained via image processing system for biological samples 280 and/or EHR system 290, and may employ pre-processing procedures involved in generating a training dataset. For example, in some implementations, data may be generated as a multi-dimensional array or vector with values representing, and to prevent the machine learning system from overemphasizing certain readings, values may be normalized to a predetermined range (e.g. 0-1, 0-100, or any other such range). The normalization may comprise linear rescaling, or may be a more complex function. In some implementations, dimension reduction may be performed to reduce large and sparse arrays or vectors. In some implementations, feature recognition may be performed to select a subset of features for further analysis, such as principal component analysis.

In various embodiments, a machine learning system 230 may be used to implement various machine learning functionality discussed herein. Machine learning system 230 may include a training engine 235 configured to train predictive models using, for example, data obtained from or via data acquisition unit 220 and/or processed data obtained from or via data analyzer 225. The training engine 235 may, for example, generate or obtain training datasets from or via data analyzer 225 and may perform validation of datasets. The training engine 235 may comprise a feature analyzer used to evaluate features by, for example, quantifying the impact of each feature on the developed model. Such a feature analyzer may, for example, uncover clinically important features that were globally predictive of the outcome, and may determine, for example, contributions of all features, or the top features (e.g., the top 2, top 5, top 10, top 15, top 20, top 25, top 30, etc.) on individual predictions. Features may be selected based on a threshold, such a percent contribution to predicting a medical condition, such as 0.5%, 1%, 2%, 5%, 10%, etc. A testing and application engine 240 may be configured to test and apply models trained via training engine 235 to, for example, study subject and/or patient data from data acquisition unit 220 and/or data analyzer 225.

In various embodiments, a transceiver 245 allows the computing device 210 to exchange readings, control commands, and/or other data with image processing system for biological samples 280 (or components thereof) and/or EHR system 290 (or components thereof). The transceiver 245 may additionally or alternatively include a network interface permitting the computing device 210 to communicate with other remote devices and systems via, for example, a telecommunications network such as the internet. One or more user interfaces 250 allow the computing device 210 to receive user inputs (e.g., via a keyboard, touchscreen, microphone, camera, etc.) and provide outputs (e.g., via a touchscreen or other display screen, audio speakers, haptic devices, etc.). A display screen may be employed, for example, to provide real time or near real time waveforms or other readings or measurements obtained via sensors being used to capture physiological data from subjects and patients. The computing device 210 may additionally include one or more databases 255 (stored in, e.g., one or more computer-readable non-volatile memory devices) for storing, for example, data and analyses obtained from or via data acquisition unit 220, data analyzer 225, machine learning system 230 (e.g., training engine 235 and/or testing and application engine 240), image processing system for biological samples 280, and/or EHR system 290. In some implementations, database 255 (or portions thereof) may alternatively or additionally be part of another computing device that is co-located or remote and in communication with computing device 210, image processing system for biological samples 280 (or components thereof), and/or EHR system 290.

In one aspect, the present disclosure provides a method for predicting prognosis in a breast cancer patient comprising applying, by a computing system having one or more processors, a trained model to detect and quantify (a) cancer cells and (b) tumor-infiltrating leukocytes (TILs) in a biomedical image of a resected tumor sample including a tumor stromal region, wherein the resected tumor sample is obtained from the breast cancer patient; computing a fractal-geometric metric based on anatomic distribution of the TILs in the biomedical image of the resected tumor sample; and determining that the breast cancer patient has a favorable prognosis when the fractal-geometric metric falls below a predetermined threshold, or determining that the breast cancer patient has a negative prognosis when the fractal-geometric metric is at or above a predetermined threshold. In some embodiments, negative prognosis comprises recurrent disease in breast tissue, bone tissue, or brain tissue. Additionally or alternatively, in some embodiments, the method further comprises providing a cancer therapy recommendation based on the fractal-geometric metric, and/or administering a cancer therapy to the breast cancer patient based on the fractal-geometric metric. Examples of suitable cancer therapy may comprise one or more of surgery, chemotherapy, immunotherapy and radiation therapy.

In one aspect, the present disclosure provides a method for selecting a breast cancer patient for treatment with a chemotherapeutic agent comprising applying, by a computing system having one or more processors, a trained model to detect and quantify (a) cancer cells and (b) tumor-infiltrating leukocytes (TILs) in a biomedical image of a resected tumor sample including a tumor stromal region, wherein the resected tumor sample is obtained from the breast cancer patient; computing a fractal-geometric metric based on anatomic distribution of the TILs in the biomedical image of the resected tumor sample; and administering a chemotherapeutic agent to the breast cancer patient, wherein the fractal-geometric metric of the breast cancer patient falls below a predetermined threshold. Examples of chemotherapeutic agents include, but are not limited to, alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, and endocrine/hormonal agents. In certain embodiments, the chemotherapeutic agent is selected from the group consisting of cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), bevacizumab, oxaliplatin, melphalan, etoposide, mechlorethamine, bleomycin, microtubule poisons, annonaceous acetogenins, or combinations thereof.

In another aspect, the present disclosure provides a method for selecting a breast cancer patient for surgery, radiation therapy, or immunotherapy comprising applying, by a computing system having one or more processors, a trained model to detect and quantify (a) cancer cells and (b) tumor-infiltrating leukocytes (TILs) in a biomedical image of a resected tumor sample including a tumor stromal region, wherein the resected tumor sample is obtained from the breast cancer patient; computing a fractal-geometric metric based on anatomic distribution of the TILs in the biomedical image of the resected tumor sample; and administering surgery, radiation therapy, or immunotherapy to the breast cancer patient, wherein the fractal-geometric metric of the breast cancer patient is at or above a predetermined threshold. In certain embodiments, the immunotherapy comprises one or more of an anti-PD-1 antibody, an anti-PD-L1 antibody, an anti-PD-L2 antibody, an anti-CTLA-4 antibody, an anti-TIM3 antibody, an anti-TIGIT antibody, an anti-VISTA antibody, an anti-B7-H3 antibody, an anti-BTLA antibody, an anti-CD73 antibody, or an anti-LAG-3 antibody. Examples of immunotherapy may include one or more of pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189.

Additionally or alternatively, in some embodiments, the breast cancer is triple negative breast cancer, HER2-positive breast cancer, Estrogen-Receptor (ER) positive breast cancer, or Progesterone-Receptor (PR) positive breast cancer. The breast cancer may be metastatic or primary. In certain embodiments, the breast cancer patient suffers from stage I cancer, stage II cancer, stage III cancer, or stage IV breast cancer. Additionally or alternatively, in certain embodiments, the biomedical image is a hematoxylin and eosin (H&E)-stained image, a radiographic image or an immunohistochemical (IHC) image. In any of the preceding embodiments of the methods disclosed herein, the tumor stromal region includes basement membrane, fibroblasts, extracellular matrix, immune cells, and mesenchymal stromal cells.

Additionally or alternatively, in some embodiments, the methods of the present technology further comprise computing an additional metric based on quantity and anatomic distribution of HER2-expressing cancer cells, and/or Trop2-expressing cancer cells in the biomedical image of the resected tumor sample.

In any and all embodiments of the methods disclosed herein, the trained model is a machine learning model generated using a machine learning technique. In some embodiments, the machine learning technique is a random forest technique, and the machine learning classification model is a random forest model. Alternatively, other machine learning classification models such as Naïve Bays, Support Vector Machines, Decision Trees, KNN (k-nearest neighbours), Generalized Linear Models, Bagging, convolutional neural networks (CNN) and the like may be used.

In one aspect, the present disclosure provides a computer system for predicting prognosis in a breast cancer patient, the computing system comprising a processor and a memory with instructions which, when executed by the processor, cause the processor to: apply, by a computing system having one or more processors, a trained model to detect and quantify (a) cancer cells and (b) tumor-infiltrating leukocytes (TILs) in a biomedical image of a resected tumor sample including a tumor stromal region, wherein the resected tumor sample is obtained from the patient; compute a fractal-geometric metric based on anatomic distribution of the TILs in the biomedical image of the resected tumor sample; and determine that the breast cancer patient has a favorable prognosis when the fractal-geometric metric falls below a predetermined threshold, or determining that the breast cancer patient has a negative prognosis when the fractal-geometric metric is at or above a predetermined threshold. In some embodiments, negative prognosis comprises recurrent disease in breast tissue, bone tissue, or brain tissue.

Additionally or alternatively, in some embodiments, the breast cancer is triple negative breast cancer, HER2-positive breast cancer, Estrogen-Receptor (ER) positive breast cancer, or Progesterone-Receptor (PR) positive breast cancer. The breast cancer may be metastatic or primary. In certain embodiments, the breast cancer patient suffers from stage I cancer, stage II cancer, stage III cancer, or stage IV breast cancer. Additionally or alternatively, in certain embodiments, the biomedical image is a hematoxylin and eosin (H&E)-stained image, a radiographic image or an immunohistochemical (IHC) image. In any of the preceding embodiments of the methods disclosed herein, the tumor stromal region includes basement membrane, fibroblasts, extracellular matrix, immune cells, and mesenchymal stromal cells.

In any and all embodiments of the computer systems disclosed herein, the trained model is a machine learning model generated using a machine learning technique. In some embodiments, the machine learning technique is a random forest technique, and the machine learning classification model is a random forest model. Alternatively, other machine learning classification models such as Naïve Bays, Support Vector Machines, Decision Trees, KNN (k-nearest neighbours), Generalized Linear Models, Bagging, convolutional neural networks (CNN) and the like may be used.

Additionally or alternatively, in some embodiments of the computer systems disclosed herein, the instructions further cause the processor to compute an additional metric based on quantity and anatomic distribution of HER2-expressing cancer cells, and/or Trop2-expressing cancer cells in the biomedical image of the resected tumor sample. Additionally or alternatively, in certain embodiments, of the computer systems disclosed herein, the instructions further cause the processor to provide a cancer therapy recommendation based on the fractal-geometric metric. Examples of cancer therapy include surgery, chemotherapy, immunotherapy and radiation therapy.

### Examples

The present technology is further illustrated by the following Examples, which should not be construed as limiting in any way.

### Example 1: Experimental Methods

Methods: We analyzed standard H&E slides for twelve cases of primary triple-negative breast cancer (TNBC) resected from patients prior to the initiation of any therapy. The term triple-negative refers to the absence of estrogen and progesterone receptors and the absence of HER2, a histologic condition associated generally with poor prognosis although paradoxically many patients are cured by modern treatment. All twelve patients were treated by standard of care primary surgery, radiation therapy, and post-operative systemic chemotherapy. Three of the twelve cases experienced eventually developed recurrent disease (ipsilateral breast, bone, brain) while nine of the twelve cases remained disease-free at prolonged follow-up durations. Hence, the initial test set has a representation of cases with both poor and good prognoses.

To count TILs we used QuPATH, an open-source software package for digital pathology image analysis.⁷ We trained a random forest classifier to detect and quantify TILs as compared with cancer cells. For each case, we examined a square area representative of the tumor, including stroma, toward the center of the mass. The side length of this larger square was 1000 μm, which we divided into sixteen equal smaller squares of 250 μm by 250 μm each, counting N, the number of TILs in each small square. Letting

$$S ( x ) = {\sum }_{t = 1}^{x} N ( i )$$

our metric is

$$D = 1 + \frac{2 [ ln S ( 16 ) - ln {N}_{max} ]}{ln ( 16 )}$$

with Nₘₐₓ being the number of TILs in small square with the highest number of TILs. Intuitively, a D=2 would be consistent with the fractal dimension of a simple sheet, while a D=3 would be consistent with the fractal dimension of a solid mass of TILs.

### Example 2: The Methods of the Present Technology Accurately Classify Breast Cancer Tumor Samples

The mean D for the three cases who recurred was 2.77 with a standard deviation of 0.0444. The Mean/for the 9 cases who remained free of disease was 2.65 with a standard deviation of 0825. This is a statistically significant difference at p<0.03 using the Student's t-test, two-sided, for populations of unequal variance. Of the cases with the top four values of D, ranging from 2.72 to 2.84, three suffered recurrent disease while of the eight cases with values of D<2.72, ranging from 2.55 to 2.70, none have recurred.

These results show that the dimensionality of TIL anatomic distribution predicts prognosis in breast cancer (e.g., triple negative breast cancer) patients. While we used digital pathology methods to count TILs, the calculation of our metric is agnostic to the method of TIL counting.

In addition to the clinical utility of TIL dimensionality, its etiology is a topic of interest. In a clinically-relevant animal model it was shown that cancer cells starting from a given site can traverse a hematogenous route to seed discrete, unconnected tumors.⁸ This phenomenon of cell mobility between sites of disease has also been confirmed in cancer xenografts by single-cell sequencing.⁹ An anatomic concurrence of TILs with such cancer cell seeds has been corroborated, albeit not quantified, as suggested in FIG. 6E of Bankhead, P. et al. Scientific Reports (2017).

Hence the quantitative anatomic arrangement of the TILs as estimated by the calculation of D may be a measure of the magnitude of self-seeding, which could have broad pragmatic implications.¹⁰ Furthermore, TILs have been shown to exert many non-immunological biological effects on cancer cells, including promoting resistance to chemotherapy.¹¹

As to Immunobiology and its prognostic and therapeutic consequences, the methods of the present technology can be applied to the study of a large clinically annotated dataset that includes immunohistochemically defined leukocyte subsets. Furthermore, these analytic methods can be applied to other countable, clinically relevant features of breast cancer including cancer cells with markers for hormone receptors, HER2, and Trop2.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1. Mandelbrot, Benoit B. The Fractal Geometry of Nature. New York: W.H. Freeman and Co., 1982. ISBM 0-7167-1186-9.
2. Bairi K, Haynes H R, Blackley E et al. The tale of TILs in breast cancer: A report from The International Immuno-Oncology Biomarker Working Group. npJ Breast Cancer. 2021; 7:150. Published online 2021 Dec. 1. doi: 10.1038/s41523-021-00346-1 PMCID: PMC8636568
3. Loi S, Michiels S, Adams S et al. The journey of tumor-infiltrating lymphocytes as a biomarker in breast cancer: clinical utility in an era of checkpoint inhibition. Ann Oncol. 2021 October; 32 (10): 1236-1244. doi: 10.1016/j.annonc.2021.07.007. Epub 2021 Jul. 24.PMID: 34311075
4. Yam C, Yen E Y, Chang J T et al. Immune Phenotype and Response to Neoadjuvant Therapy in Triple-Negative Breast Cancer. Clin Cancer Res. 2021 Jul. 12. doi: 10.1158/1078-0432.CCR-21-0144. Online ahead of print. PMID: 34253579
5. Amgad M, Stovgaard E S, Balslev E, et al. for the International Immuno-Oncology Biomarker Working Group. Report on Computational Assessment of Tumor Infiltrating Lymphocytes from the International Immuno-Oncology Biomarker Working Group. npj Breast Cancer. 2020 May 12; 6:16. doi: 10.1038/s41523-020-0154-2. PMID: 32411818.
6. Schroeder, Manfred. Fractals, Chaos, Power Laws: Minutes from an Infinite Paradise. New York: W.H. Freeman and Co., 1991. p. 215-220. ISBM 0-7167-2136-8.
7. Bankhead, P. et al. QuPath: Open source software for digital pathology image analysis. Scientific Reports (2017). https://doi.org/10.1038/s41598-017-17204-5.
8. Kim M-Y, Oskarsson T, Acharyya S, Nguyen D X, Zhang X H-F, Norton L, Massage J. Tumor self-seeding by circulating cancer cells. Cell. 2009 Dec. 24; 139 (7). 1315-26. doi: 10.1016/j.cell.2009.11.025. PMID: 20064377.
9. Quinn J J, Jones M G, Okimoto R A, Nanjo S, Chan M M, Yosef N, Bivona T G. Weissman J S. Single-cell lineages reveal the rates, routes, and drivers of metastasis in cancer xenografts. Science. 2021 Feb. 26; 371 (6532): eabc1944. doi: 10.1126/science.abc1944. PMID: 33479121.
10. Comen E, Norton L, Massagué J, Clinical implications of cancer self-seeding. Nat Rev Clin Oncol. 2011 June; 8 (6): 369-77. doi: 10.1038/nrclinonc.2011.64. Epub 2011 Apr. 26. PMID: 21522121.
11. Acharyya S, Oskarsson T, Vanharanta S, Malladi S, Kim J, Morris P G, Manova-Todorova K, Leversha M, Hogg N, Seshan V E, Norton L, Brogi E, Massagué J. A CXCL1 paracrine network links cancer chemoresistance and metastasis. Cell. 2012 Jul. 6; 150 (1): 165-78. doi: 10.1016/j.cell.2012.04.042.PMID: 22770218
12. Oshi M, Katsuta E, Yan L et al, A Novel 4-Gene Score to Predict Survival, Distant Metastasis and Response to Neoadjuvant Therapy in Breast Cancer. Cancers (Basel). 2020 May 2; 12 (5): 1148. doi: 10.3390/cancers12051148.PMID: 32370309

1. (canceled)

2. (canceled)

3. A method for selecting a breast cancer patient for treatment with a chemotherapeutic agent comprising
applying, by a computing system having one or more processors, a trained model to detect and quantify (a) cancer cells and (b) tumor-infiltrating leukocytes (TILs) in a biomedical image of a resected tumor sample including a tumor stromal region, wherein the resected tumor sample is obtained from the breast cancer patient;
computing a fractal-geometric metric based on anatomic distribution of the TILs in the biomedical image of the resected tumor sample; and
administering a chemotherapeutic agent to the breast cancer patient, wherein the fractal-geometric metric of the breast cancer patient falls below a predetermined threshold.

4. The method of claim 3, wherein the chemotherapeutic agent comprises one or more agents selected from the group consisting of alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, and endocrine/hormonal agents; or
wherein the chemotherapeutic agent is selected from the group consisting of cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), bevacizumab, oxaliplatin, melphalan, etoposide, mechlorethamine, bleomycin, microtubule poisons, annonaceous acetogenins, or combinations thereof.

5. (canceled)

6. A method for selecting a breast cancer patient for surgery, radiation therapy, or immunotherapy comprising
applying, by a computing system having one or more processors, a trained model to detect and quantify (a) cancer cells and (b) tumor-infiltrating leukocytes (TILs) in a biomedical image of a resected tumor sample including a tumor stromal region, wherein the resected tumor sample is obtained from the breast cancer patient;
computing a fractal-geometric metric based on anatomic distribution of the TILs in the biomedical image of the resected tumor sample; and
administering surgery, radiation therapy, or immunotherapy to the breast cancer patient, wherein the fractal-geometric metric of the breast cancer patient is at or above a predetermined threshold.

7. The method of claim 6, wherein the immunotherapy comprises one or more of an anti-PD-1 antibody, an anti-PD-L1 antibody, an anti-PD-L2 antibody, an anti-CTLA-4 antibody, an anti-TIM3 antibody, an anti-TIGIT antibody, an anti-VISTA antibody, an anti-B7-H3 antibody, an anti-BTLA antibody, an anti-CD73 antibody, or an anti-LAG-3 antibody; or
wherein the immunotherapy comprises one or more agents selected from the group consisting of pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189.

8. (canceled)

9. The method of claim 3, wherein the breast cancer is triple negative breast cancer, HER2-positive breast cancer, Estrogen-Receptor (ER) positive breast cancer, or Progesterone-Receptor (PR) positive breast cancer or wherein the breast cancer is metastatic or primary.

10. The method of claim 3, wherein the biomedical image is a hematoxylin and eosin (H&E)-stained image, a radiographic image or an immunohistochemical (IHC) image.

11. The method of claim 3, further comprising computing an additional metric based on quantity and anatomic distribution of HER2-expressing cancer cells, and/or Trop2-expressing cancer cells in the biomedical image of the resected tumor sample.

12. The method of claim 3, wherein the breast cancer patient suffers from stage I cancer, stage II cancer, stage III cancer, or stage IV breast cancer.

13. (canceled)

14. The method of claim 3, wherein the trained model is a machine learning model generated using a machine learning technique, optionally wherein the machine learning technique is a random forest technique, and wherein the machine learning model is a random forest model.

15. (canceled)

16. The method of claim 3, wherein the tumor stromal region includes basement membrane, fibroblasts, extracellular matrix, immune cells, and mesenchymal stromal cells.

17. A computer system for predicting prognosis in a breast cancer patient, the computing system comprising a processor and a memory with instructions which, when executed by the processor, cause the processor to:
apply, by a computing system having one or more processors, a trained model to detect and quantify (a) cancer cells and (b) tumor-infiltrating leukocytes (TILs) in a biomedical image of a resected tumor sample including a tumor stromal region, wherein the resected tumor sample is obtained from the patient;
compute a fractal-geometric metric based on anatomic distribution of the TILs in the biomedical image of the resected tumor sample; and
determine that the breast cancer patient has a favorable prognosis when the fractal-geometric metric falls below a predetermined threshold, or determining that the breast cancer patient has a negative prognosis when the fractal-geometric metric is at or above a predetermined threshold.

18. The computer system of claim 17, wherein negative prognosis comprises recurrent disease in breast tissue, bone tissue, or brain tissue.

19. The computer system of claim 17, wherein the breast cancer is triple negative breast cancer, HER2-positive breast cancer, Estrogen-Receptor (ER) positive breast cancer, or Progesterone-Receptor (PR) positive breast cancer or wherein the breast cancer is metastatic or primary.

20. The computer system of claim 17, wherein the biomedical image is a hematoxylin and eosin (H&E)-stained image, a radiographic image or an immunohistochemical (IHC) image.

21. The computer system of claim 17, wherein the instructions further cause the processor to compute an additional metric based on quantity and anatomic distribution of HER2-expressing cancer cells, and/or Trop2-expressing cancer cells in the biomedical image of the resected tumor sample.

22. The computer system of claim 17, wherein the breast cancer patient suffers from stage I cancer, stage II cancer, stage III cancer, or stage IV breast cancer.

23. (canceled)

24. The computer system of claim 17, wherein the trained model is a machine learning model generated using a machine learning technique, optionally wherein the machine learning technique is a random forest technique, and wherein the machine learning model is a random forest mode.

25. (canceled)

26. The computer system of claim 17, wherein the tumor stromal region includes basement membrane, fibroblasts, extracellular matrix, immune cells, and mesenchymal stromal cells.

27. The computer system of claim 17, wherein the instructions further cause the processor to provide a cancer therapy recommendation based on the fractal-geometric metric.

28. The computer system of claim 27, wherein the cancer therapy may comprise one or more of surgery, chemotherapy, immunotherapy and radiation therapy.
